(19) United States
(12) Patent Application Publication
(10) Pub. No.: US 2010/0154793 A1
(43) Pub. Date: Jun. 24, 2010

(54) MEDICINE EJECTION DEVICE

(75) Inventors: Masaya Kobayashi, Yokohama-shi (JP); Mitsuru Imai, Chichibu-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/375,929

(22) PCT Filed: Jul. 30, 2007
(86) PCT No.: PCT/JP2007/065312

§ 371 Date: Jan. 30, 2009

(30) Foreign Application Priority Data
Aug. 1, 2006 (JP) 2006-209365
Jul. 26, 2007 (JP) 2007-193998

(51) Int. Cl. A61M 15/00

(52) U.S. Cl. 128/203.14

(57) ABSTRACT

This medicine ejection device is directed at uniformizing an amount of a medicine inhaled by a user through a mouthpiece: and ejects a predetermined constant amount of the medicine by measuring a remaining amount of the medicine in a reservoir 7 at every inhalation time with the use of a strain gauge 37, which supplies the medicine through a connection tube 8b to an ejection head 8a for ejecting a medicine into a flow path 20 of a mouthpiece 4, so as to compensate for a decrease of an amount to be inhaled due to the deposition of the medicine in a nozzle (orifice) of the ejection head 8a, and by adjusting an ejecting operation duration of the ejection head 8a when the user inhales the medicine next time.

### TECHNICAL FIELD

The present invention relates to a medicine ejection device for ejecting a medicine to be inhaled by a user including an inhaler or the like.

### BACKGROUND ART

A medicine ejection device can optimally treat a user while using an information database such as an electronic chart. The medicine ejection device has a memory unit for storing information on an individual user including the information of the medical chart and a prescription for the user, and an ejection unit for ejecting a medicine as fine liquid droplets. For instance, International Publication WO95/01137 and International Publication WO02/04043 disclose a medicine ejection device having an ejection control unit which makes the device eject a medicine while controlling an inhaler corresponding to an inspiratory profile, so that the user can inhale the medicine according to the information of the prescription.

Such a medicine ejection device ought to efficiently administer a medicine to an individual user, concurrently with precisely controlling an applied dose of the medicine and a dosing interval.

For this reason, a method was invented which includes ejecting a medicine in a form of droplets having an appropriate size with a predetermined number into an airflow to be inhaled through a mouthpiece, by using an ejection principal of an ink-jet system. The method also can uniformize the particle size. However, in order to make the medicine to be inhaled into a body, it is important for the droplets to have such a very small size as several micrometers. For this reason, an orifice of an ejection head is also required to have a diameter with a size of several micrometers. Then, when a medicine is inhaled several times through the same ejection head, the medicine remaining in a previous ejection deposits on an inner wall of some orifices (nozzles) and easily blocks the orifice. Then, there are a smaller number of ejectable orifices left in the next time of inhalation, so that an amount of the medicine ejected by driving the ejection head for the same period as in the previous time was occasionally smaller than the predetermined amount to be ejected.

In addition, there is a case where a medicine cannot sufficiently fill a liquid room of an ejection head, if the surface of an inner wall of a liquid room is not made adequately wettable when the medicine is charged into the liquid room. Accordingly, an ejected amount tends to be less than an expected amount in an early state of ejection. In an ejection head of a normal printer or the like using thermal energy or piezoelectric energy, the problem is circumvented by ageing the ejection head to sufficiently make the surface of the ejection element wettable to ink and then charging the ink in the ejection head before shipping.

On the other hand, an inhaler is a device for making a user inhale a medicine, so that the medicine is charged into the ejection unit just before inhalation so as to prevent the medicine from being denatured. The medicine contacts with the surface of the inner wall of a liquid room firstly at the time, which tends to cause the above described problem. As a result of this, the inhaler cannot eject a predetermined amount of a medicine, and hardly makes a user inhale the predetermined amount of the medicine.

In other words, when ejecting the medicine by driving an ejection head constantly on a driving condition determined at the beginning while assuming that the medicine ejecting amount per unit time is constant throughout ejecting operation duration, the predetermined amount of the medicine (normally determined by a doctor who prescribes the medicine) may not be actually ejected. This is because the medicine ejecting amount per unit time is affected by the above described factor, and does not become constant even though the medicine is ejected on the constant driving condition.

The problem does not occur if it would be possible to precisely measure the amount of the medicine ejected from the ejection head in-situ, and to stop driving an ejection head after the predetermined amount of the medicine has been ejected. However, this is difficult under the present conditions.

Thus, a medicine ejection device has an unsolved problem that a user cannot inhale a required amount of the medicine, because the preset ejection amount is not ejected from the orifice; and is actually difficult to be practically used.

### DISCLOSURE OF THE INVENTION

The present invention is designed with respect to the above described unsolved problem of a conventional technology, and is directed at providing a medicine ejection device which stabilizes a medicine ejecting amount ejected into a flow path of a mouthpiece and can adequately control a dose of the medicine to be applied to a user.

In view of the above described problem, a medicine ejection device for ejecting a medicine to be inhaled by a user according to the present invention comprises: a medicine ejection portion for ejecting the medicine; a measurement portion for measuring a medicine ejecting amount ejected from the medicine ejection portion; and a controller for driving the medicine ejection portion so as to eject the amount of the medicine, which corresponds to a difference between the amount of the medicine to be administered and the medicine ejecting amount, on the basis of a value measured in the measurement portion.

A medicine ejection device according to the present invention can eject an applied dose of a medicine to be inhaled by a user more precisely and more reliably than ever. For instance, the medicine ejection device can maintain a stable medicine ejecting amount by measuring a medicine ejecting amount in the previous inhaled time, and adjusting an ejecting operation duration in the subsequent steps, and thereby can control a dose of the medicine applied to the user appropriately.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a perspective appearance view illustrating an inhaler which is one embodiment (Exemplary embodiment 1) of a medicine ejection device according to the present invention.

FIG. 2 is a perspective appearance view illustrating a device in FIG. 1, in a state of which the access cover is opened.

FIG. 3 is a perspective appearance view illustrating only a medicine ejection unit of a device in FIG. 1.

FIG. 4 is a sectional view illustrating a state of a device in FIG. 1, before being filled with a medicine;

FIG. 5 is a sectional view illustrating a state of a device in FIG. 1, after having been filled with a medicine.

FIG. 6 is a flow chart illustrating an inhalation step according to Exemplary embodiment 1.

FIG. 7 is a sectional view illustrating a state of a device of Exemplary embodiment 2, before being filled with a medicine.

FIG. 8 is a sectional view illustrating a state of a device in FIG. 7, after having been filled with a medicine.

FIG. 9A is a view for describing an action of a measurement portion in a device in FIG. 7, which optically measures an ejected amount per unit time.

FIG. 9B is a view for describing an action of a measurement portion in a device in FIG. 7, which optically measures an ejected amount per unit time.

FIG. 10 is a view illustrating one variation of Exemplary embodiment 2.

FIG. 11 is a flow chart illustrating inhalation steps in Exemplary embodiment 2.

FIG. 12 is a view illustrating a circuit configuration of an ejection correction section.

### BEST MODE FOR CARRYING OUT THE INVENTION

Preferred embodiments of the present invention will now be described in detail in accordance with the accompanying drawings.

### Exemplary Embodiment 1

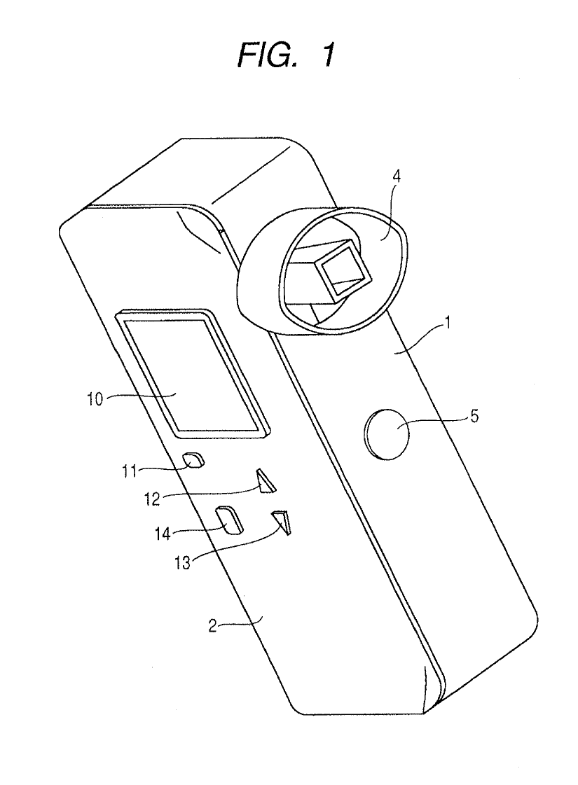

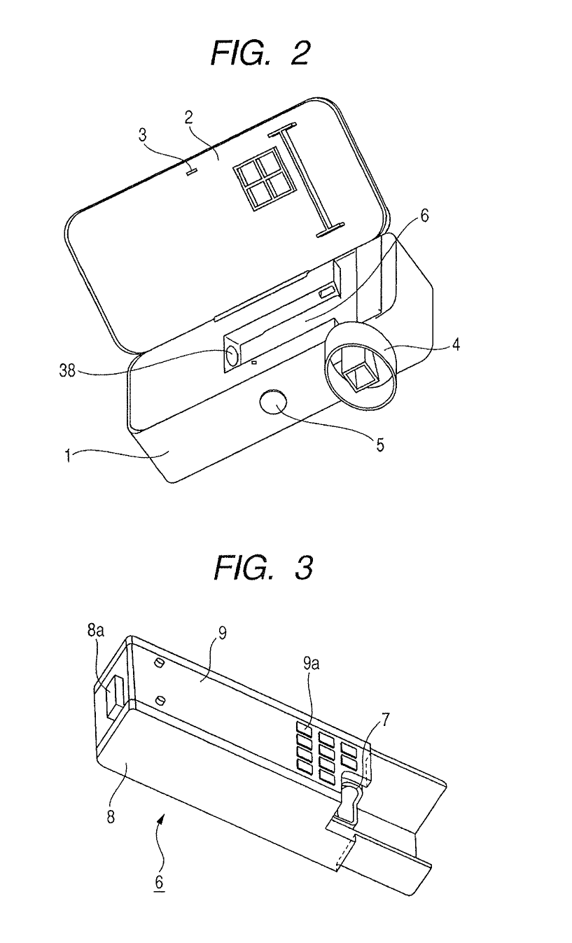

FIG. 1 is a perspective appearance view illustrating an inhaler which is one example of a medicine ejection device according to the present invention. A main body of the inhaler has an outer jacket formed of a housing 1 and an access cover 2. The access cover 2 is provided with a hook member 3 for locking as shown in FIG. 2. A mouthpiece 4 is removably attached to the housing 1.

The hook member 3 of the access cover 2 is configured so as to be locked on a hook shaft which works together with a lock release button 5 pushed by a spring. When the access cover 2 is opened, the lock release button 5 is pushed. Thereby, a lock of a hook is unhooked and the access cover 2 is opened by the force of the spring which works in a direction for the access cover 2 to open. The access cover 2 has a display unit 10 installed thereon so as to display an applied dose, the time of day and an error message. The access cover 2 also has a menu change button 11, an upward button 12 of a set button, a downward button 13 and a determination button 14 installed so that a user can set the dose and the like.

FIG. 2 illustrates the state of the inhaler of which the access cover 2 is opened. When the access cover 2 is opened, a medicine ejection unit 6 integrated with an ejection portion and a reservoir and a mouthpiece 4 appear. The medicine ejection unit 6 is removable from a device, and is attached/detached every time before/after the inhalation, or is replaced when only a little amount of the medicine remains after a plurality of the inhalations.

The medicine ejection unit 6 is provided with a reservoir 7 containing a medicine in a flexible container, an ejection head portion 8 of a medicine ejection portion for ejecting the medicine, and an electric connection member 9 having an electric connection surface 9a for supplying an electric power for generating thermal energy to a heater installed in the ejection head portion 8, as is shown in FIG. 3. A battery rechargeable as a secondary battery is held inside a main body of an inhaler and supplies electricity to the electric connection surface 9a.

In FIG. 3, the reservoir 7 for storing the medicine therein and the ejection head 8 for ejecting the medicine are integrated to form a medicine ejection unit 6 having a cartridge shape, but the reservoir 7 and the ejection head 8 may be separately arranged.

The medicine ejection portion (ejection head portion 8) according to the present invention can have an arbitrary ejection energy generating element. In other words, the medicine ejection portion can employ any of ejection principles including a powder ejection type, an MDI type, a jet-type nebulizer, an ultrasonic wave type nebulizer, a mesh type nebulizer, an extrusion system using a cam, and an ink-jet system, which are not exclusive. The above described ink-jet system is taken in a broad sense, and includes the case of ejecting a medicine. The ejection energy generating element can employ an electrothermal transducer for applying thermal energy to the medicine, or an electromechanical transducer for applying mechanical energy, for instance. In other words, a method for ejecting the medicine includes a method of ejecting the medicine through an ejection nozzle by applying the thermal energy (thermal jet type) with the use of the electrothermal transducer, and a method of ejecting the medicine through the ejection nozzle by applying the mechanical energy to the medicine with the use of a vibratory pressure generated from the electromechanical transducer (such as a piezoelectric element), for instance. The ejection method can be selected according to a type of the medicine and the like.

When the thermal jet type is employed, the individual medicine ejection unit can show a high size-accuracy of a bore diameter of the ejection nozzle, a heat quantity of a heat pulse to be used for ejection and a micro heater of the electrothermal transducer, and a high reproducibility of ejection. Accordingly, the thermal jet type can achieve a narrow distribution of droplet sizes. The thermal jet type also has a low manufacturing cost for the head and can be sufficiently applied to a small device which needs to replace the head frequently. Accordingly, when being required to be portable or convenient, the medicine ejection device can employ particularly the thermal jet type of an ejection device.

The medicine to be used in the present invention is a notion including not only the medicine of a medicinal compound showing a pharmacologic and physiological action, but also a component for scenting or flavoring, a dye and a pigment. The medicine may be a liquid or a powder.

The liquid medicine to be used in the present invention means a medicine in a liquid form or a liquid medium including a medicine. The medicine may also include an arbitrary additive. The medicine may form any of solution, dispersion, emulsion, suspension, or slurry in a liquid, and had better be uniformized in the liquid.

When a liquid medicine is used as a medicine, the main medium of the liquid can be water or an organic matter, but can rather be water in consideration of being administrated to a living body.

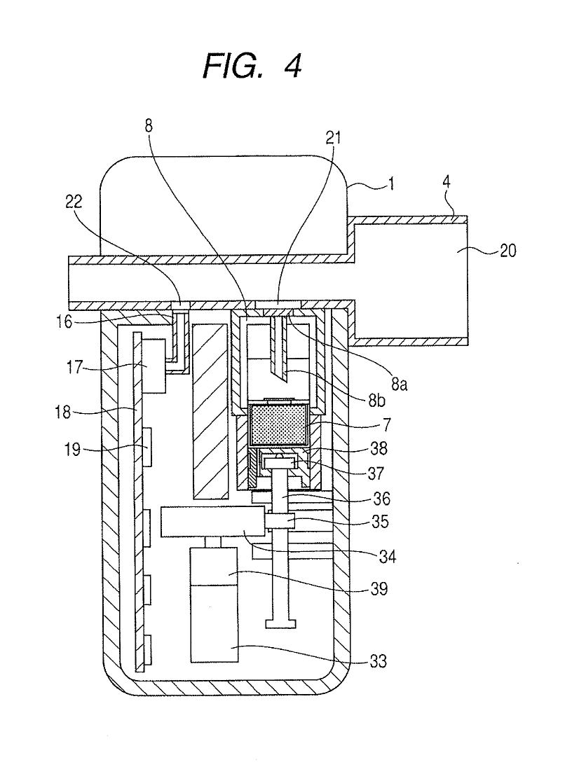

FIG. 4 illustrates a cross section of a device in FIG. 1. In the figure, a mouthpiece 4 has an air flow path 20 for inhalation and an opening 21 for mixing droplets ejected from a medicine ejection unit 6 with an inhalation airflow in the air flow path 20. An end of the air flow path 20 in the opposite side to the mouthpiece 4 is an air inlet for intaking outer air so as to produce an airflow when a user has inhaled a medicine. The inhaler had better synchronize the inhalation by the user with the ejection of the droplets so that the user can inhale the medicine effectively. For the purpose, the inhaler had better detect the inhalation of the user and start the ejection in response to an inhalation sensing signal, and accordingly has a pressure sensor 17 in a control substrate (controller) 18, as a sensor for detecting a negative pressure in the air flow path 20 generated by the inhalation of the user. The mouthpiece 4 also has a communication hole 22 which makes the pressure sensor 17 communicate with the air flow path 20. The communication hole 22 communicates with the pressure sensor 17 through a pressure detecting nozzle 16.

A control substrate 18 has an inclination sensor 19 thereon which uses a tri-axial acceleration system. The inclination sensor 19 is used for correcting a remaining amount of a medicine so as to enhance an accuracy of measurement, and is also used for making a user inhale the medicine in an adequate posture. When the inclination sensor 19 indicates a result of having detected abnormality, the inhaler can display the abnormality in posture on a display unit 10 arranged on an access cover 2, and inform the abnormality to a user by using a sound, a vibration sent from a vibrating motor, or illumination by LED. Furthermore, at least the control substrate 18 has a RAM and flash ROM for storing prescription data, a ROM for storing an operation program of the inhaler, and a CPU for controlling the inhaler on the basis of the data in the ROM and the RAM arranged thereon. The control substrate 18 of a controller calculates an ejection condition including an ejecting operation duration which will be described later, and controls driving for an ejection head.

A reservoir 7 is not connected with an ejection head portion 8 in a medicine ejection unit 6 before the medicine ejection unit 6 is attached to the main body of an inhaler. This is for the purpose of preventing the deterioration of a medicine, and a result of having considered the safety of the medicine. The reservoir 7 has a thin film such as aluminum foil bonded in its ejection head portion side to prevent the medicine from leaking out from the reservoir 7. The ejection head portion 8 has an ejection head (ejection unit) 8a having a heater, a liquid room and a nozzle (orifice). A connection tube 8b for charging the medicine into the ejection head 8a has a tip of a peaky shape so as to penetrate the thin film of the reservoir 7 and charge the medicine. A thick gum plate may be used instead of the thin film, because the plate or the film has only to prevent a leakage of the liquid and make the reservoir 7 communicate with the ejection head 8a. When the thick gum plate is employed, the communication tube 8b can be made from stainless steel and has a thin shape like a needle. The structure can allow the communication tube 8b to be penetrated and extracted several times. When the inhaler is used in the next time after a long period, the medicine must be prevented from contacting with air. In such a case, the structure is very useful.

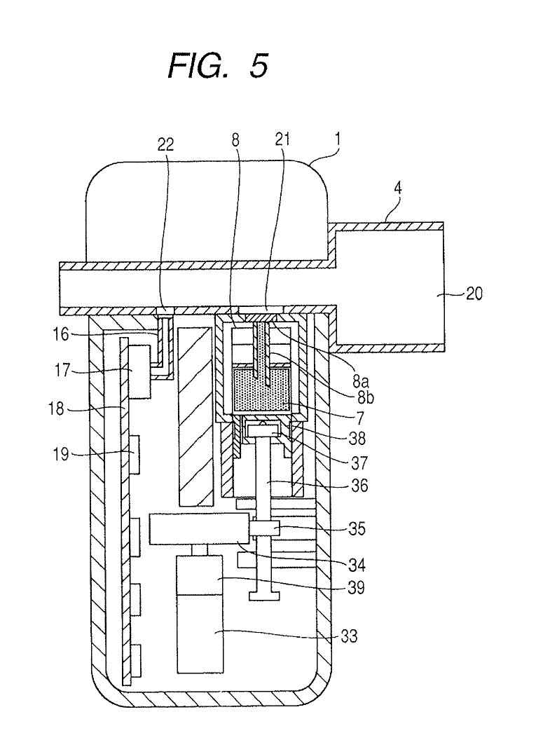

An elevating compression motor 33 arranged in a housing 1 rotates a motor gear 34 in order to make a reservoir 7 approach a communication tube 8b of an ejection head portion 8. Then, a driven gear 35 rotates. The driven gear 35 has a screw-shaped groove formed in an inner diameter side. A screw shaft 36 is arranged so as to be engaged with the screw-shaped groove. A strain gauge (remaining amount measuring means) 37 which is a measurement portion is placed in a tip of the screw shaft 36. The screw shaft 36 pushes the reservoir 7 through a reservoir elevating portion 38 which is integrally formed with the screw shaft 36 so as to surround the strain gauge 37, and connects the reservoir 7 with the communication tube 8b of an ejection head portion 8. A controller monitors a load applied to the strain gauge 37 at this time, and makes the screw shaft 36 charge the medicine into the ejection head 8a. FIG. 5 illustrates the state in which the medicine has been completely charged.

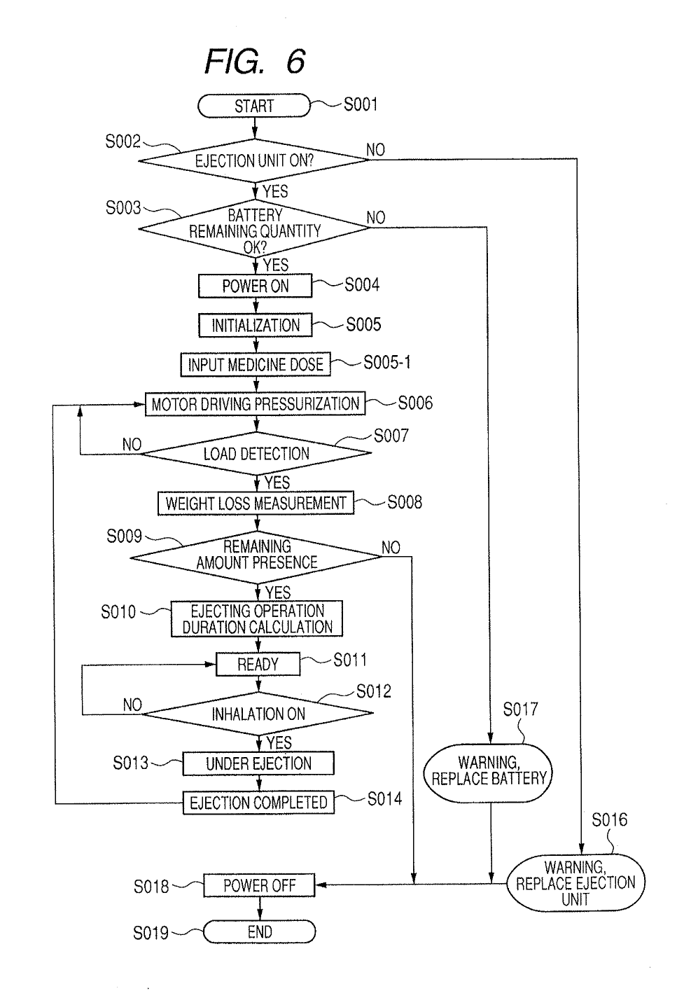

An action of the inhaler according to the present embodiment will be now described with reference to the flow chart illustrated in FIG. 6.

In a step S001 (START), the inhaler is set at a state of being capable of starting to be used by an operation of turning an electric power switch on by a user. After the starting state, the inhaler examines whether a medicine ejection unit 6 is inserted therein or not (S002: EJECTION UNIT ON?). When the medicine ejection unit 6 is not inserted therein, the inhaler displays an alarm for informing the user the absence of the medicine ejection unit 6 (S016: WARNING, REPLACE EJECTION UNIT), turns the power source off (S018: POWER OFF), and finishes its action (S019: END).

When a medicine ejection unit 6 employs, for instance, a thermal jet type for ejecting a medicine, a method of measuring an ohmic value of a heater which is an ejection energy generation unit can be employed as a detecting unit of the medicine ejection unit 6.

When the detecting unit has detected the presence of a medicine ejection unit 6, the inhaler checks a remaining quantity of a battery in the main body of the device (S003: BATTERY REMAINING QUANTITY OK?). When the remaining quantity is short, the inhaler displays a message for urging the replacement or charging of the battery (S017: WARNING, REPLACE BATTERY), turns the power source off (S018: POWER OFF), and finishes its action (S019: END). When the inhaler has determined that the remaining quantity of the battery is sufficient at least for one inhaling action, the user turns the power source on (S004: POWER ON), and sets initial conditions (S005: INITIALIZATION).

After having finished setting the initial conditions, the user may be required to input a dose of a medicine by oneself (S005-1: INPUT MEDICINE DOSE). Normally, the dose in a prescription data by a doctor is automatically set in the inhaler, but the user may change the does, for instance of insulin in consideration of a caloric intake and calorie consumption when the user inhales the medicine.

An elevation compression motor 33 drives a motor gear 34, a driven gear 35 and a screw shaft 36 in such a direction as to move a reservoir 7 to an ejection head 8a side. Then, the reservoir 7 is pushed up by a reservoir elevating portion 38 to move to the ejection head 8a side. When the reservoir 7 moves, a connection tube 8b penetrates a thin film stuck on the reservoir 7, and the reservoir 7 starts charging a medicine into the ejection head section 8. The elevation compression motor 33 further moves to charge the medicine into a nozzle of the ejection head 8a, which is a charging step. Because the nozzle has a bore diameter of several microns, a load applied on a strain gauge 37 greatly changes when the nozzle is filled with a medicine. The inhaler monitors the change. When having detected the change, the inhaler determines that the ejection head 8a has been completely filled with the medicine (S006: MOTOR DRIVING PRESSURIZATION and S007: LOAD DETECTION).

Subsequently, the inhaler measures a weight loss of a medicine (S008: WEIGHT LOSS MEASUREMENT). In this step, the inhaler measures a remaining amount of a medicine in a reservoir 7 (remaining medicine amount) and calculates the weight loss due to ejection, namely, a medicine ejecting amount by comparing the measured remaining amount with a value of the remaining amount measured last time. In other words, remaining amount measuring means (strain gauge 37) in the present embodiment corresponds to a measurement portion for measuring the medicine ejecting amount ejected through an ejection head 8a. Additionally, a rotary encoder 39 which is connected to an elevation compression motor 33 is not indispensable constitution for detecting a remaining amount of the medicine. However, the setting the rotary encoder 39 can be detecting a position of the reservoir elevating portion 38. Accordingly, the reservoir elevating potion 38 can be pushed up at an arbitrary prescribed amount, and if in such case a load of the strain gauge 37 is not changed, it can be detected that the reservoir elevating mechanism from the elevation compression motor 33 to the screw shaft 36 is considered as abnormal.

In the case of a small portable inhaler, a main body of the device may be inclined when a strain gauge 37 measures a load. In such a case, the device corrects the weight of the medicine on the basis of the inclination of the main body of the device to improve an accuracy of measurement for a remaining amount of the medicine. Specifically, a reservoir 7 is arranged above the strain gauge 37, and the more does the main body of the device incline from a vertical direction, the more does a weight component decrease; accordingly, the strain gauge 37 outputs a lower value according to an inclining degree of the main body, even though the ejection head 8a is filled with the medicine; and then the inhaler adds the weight considering the inclination with respect to the vertical direction to correct the decrement due to the inclination.

A medicine ejection device according to the present invention is characterized in that the device drives an ejection head so as to eject a medicine in an amount corresponding to a difference between the amount of the medicine to be administered and the ejected amount of the medicine, on the basis of a measured value in a measurement portion. As was described above, the amount of the medicine to be administered is inhaled in separated two times, which is a specific example for administering a constant medicine ejecting amount. The example will be now described. The amount of the medicine to be administered is the dose of the medicine taken by a user through a series of inhalations. The value is set and input by the user or a doctor as described above. The amount is occasionally referred to as a total dose in the following description. At first, the inhaler detects the remaining amount (S008: WEIGHT LOSS MEASUREMENT) and calculates a weight loss in comparison with the previous remaining amount (S009: REMAINING AMOUNT PRESENCE). This case is the first time of inhalation, so that the weight loss is zero. Next, the inhaler calculates ejecting operation duration for ejecting half the dose of the total dose (S010: EJECTING OPERATION DURATION CALCULATION), and waits for the start of the inhalation (S011: READY). In the above description, “ejecting operation duration” means a period of time after the first pulse has been applied to an ejection energy generating element and before the final pulse is applied, in a necessary period for one inhalation operation, namely, means a period in which a pulse train for generating ejection energy is supplied.

When having detected the inhalation, the inhaler carries out ejection (S012: INHALATION ON). At this time, the inhaler displays a message of being under ejection (S013: UNDER EJECTION). The inhaler may have a unit of informing the message to a user by vibration generated by a vibrating motor and/or sound. When having finished the ejection for ejecting operation duration calculated as a first inhalation, the inhaler stops the ejection (S014: EJECTION COMPLETED). Next, the inhaler returns to a motor drive compression step of a step s006, pressurizes a reservoir 7, and detects a remaining amount (S008: WEIGHT LOSS CALCULATION). The remaining amount of the medicine should be about a half, but may be different from a planned weight loss because the first medicine ejecting amount varies affected by the state of an ejection head 8a. The ejected amount per unit time of the first inhalation can be calculated by dividing the amount of really ejected medicine by the calculated ejecting operation duration. Then, the inhaler calculates the second ejecting operation duration to be ejected through the ejection head 8a (S010: EJECTING OPERATION DURATION CALCULATION) so as to eject the medicine in such an amount as to satisfy the total dose when the first inhaled amount is added. Specifically, the second ejecting operation duration is calculated by (total dose-really applied dose of medicine in first time)/(ejected amount per unit time in first time), because it is expected that the medicine will be ejected in the second ejection at approximately the same pace of an ejection amount per unit time as in the first ejection. Subsequent steps are the same steps as in the first time. Thereby, the inhaler can control an ejection unit so that the unit can compensate an error on the medicine ejecting amount, specifically, can keep the total ejection amount constant. Then, the inhaler checks the medicine remaining amount of the third time check step (S009: REMAINING AMOUNT PRESENCE). When the remaining amount is zero in the step, namely, the weight loss of the medicine is equal to or more than the dose, the inhaler considers that any more medicine to be applied does not remain, memorizes the medicine remaining amount in a flash ROM on a control substrate 18, turns the power source off (S018: POWER OFF), and finishes the action (S019: END).

When having determined that a medicine to be applies still remains, the inhaler calculates ejecting operation duration in a step S010, and the user shall inhale the medicine again. The inhaler may have such a structure as to allow the user to set the number of inhalations in accordance with one's own pulmonary capacity in the step S005-1, when there are variations of a pulmonary capacity among individuals of children, old men and sex.

In the above description, the inhaler is set so as to eject ½ of the total dose in the first inhalation, but the first ejection amount is not limited to this. For instance, the inhaler may be set so as to eject ⅔ of the total dose in the first inhalation. In this case, the inhaler calculates an ejection speed from a weight loss (corresponding to about ⅔ of a weight of the total dose) due to the first inhalation and the ejecting operation duration, and calculates the ejecting operation duration for the next dosage of about ⅓ on the basis of the speed. The inhaler can eject ½ or more of the total dose in the first inhalation, in order to eject the total dose with higher accuracy.

The inhaler may separate its ejection action into three or more times according to a similar principle. The embodiment will be now described below with the use of specific numeric values. The total dose was set at 21 μL. The inhaler calculated the ejecting operation duration to be 1.2 seconds and a drive frequency to be 10 kHz, so as to eject 10.5 L in the first inhalation (S010: EJECTING OPERATION DURATION CALCULATION). The inhaler ejected the medicine for 1.2 seconds, and then measured the medicine ejecting amount with a strain gauge 37 (S008: WEIGHT LOSS CALCULATION). The actually ejected amount was 9 μL. In other words, the inhaler ejected the medicine at 9/1.2=7.5 (μL/sec) which is a medicine ejecting amount per unit time. Then, the inhaler calculated ejecting operation duration for the second time according to the following calculation equation (S010: EJECTING OPERATION DURATION CALCULATION).

(21−9)/7.5=1.6 sec

As a result of having driven an ejection head through a controller so as to eject for 1.6 seconds, the inhaler could surely eject the total dose with high accuracy.

### Embodiment 2

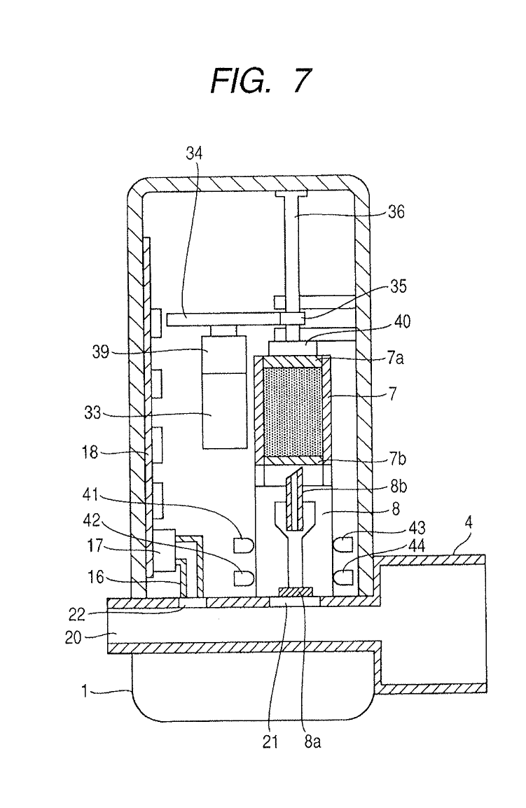

FIG. 7 illustrates a cross section of an inhaler according to Embodiment 2. An inhaler according to the present embodiment employs projectors 41 and 42 and optical receivers 43 and 44 that are a speed measuring unit for optically measuring an ejection speed (ejection amount per unit time) of a medicine ejection unit 6, in place of a strain gauge 37 for measuring a remaining amount of a medicine in a reservoir 7 as a medicine ejecting amount in one inhalation by a user. Except the above point, the inhaler has the same configuration as in the case of Embodiment 1.

A mouthpiece 4 has an opening 21 for mixing droplets ejected from a medicine ejection unit 6 with an inhalation airflow in the mouthpiece 4. The inhaler had better synchronize the inhalation by the user with the ejection of the droplets so that the user can inhale the medicine effectively. For the purpose, the inhaler had better detect the inhalation of the user and start the ejection in response to an inhalation detecting signal, and accordingly has a pressure sensor 17 as an inhalation detecting sensor in a control substrate 18. The mouthpiece 4 also has a communication hole 22 which communicates with the air flow path 20 for an inhalation. The communication hole 22 communicates with the pressure sensor 17 through a pressure detecting nozzle 16.

A reservoir 7 is not connected with an ejection head portion 8 in a medicine ejection unit 6 before the medicine ejection unit 6 is attached to the main body of an inhaler. This is for the purpose of preventing the deterioration of a medicine, and a result of having considered the safety of the medicine.

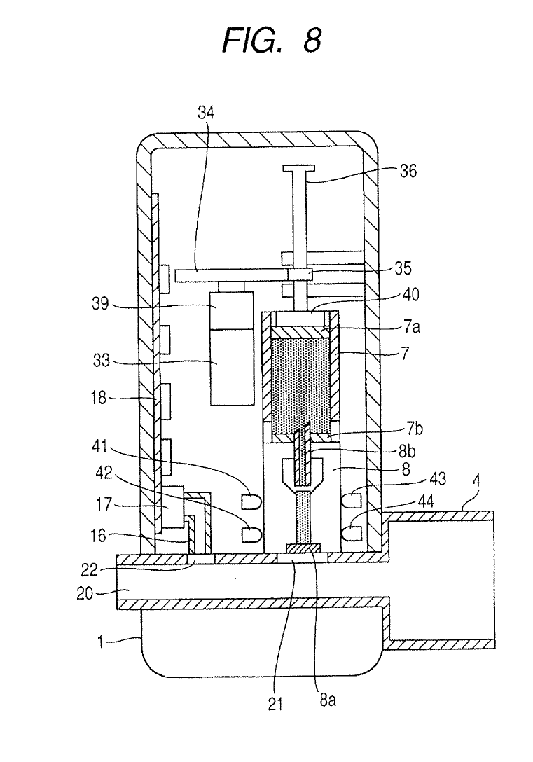

An elevating compression motor 33 rotates a motor gear 34 in order to make a reservoir 7 approach a communication tube 8b of an ejection head portion 8. Then, a driven gear 35 rotates. The driven gear 35 has a screw-shaped groove formed in an inner diameter side. A screw shaft 36 is arranged so as to be engaged with the screw-shaped groove. In a tip of the screw shaft 36, a plate 40 for pressing a packing 7a of the reservoir 7 is arranged. The elevation compression motor 33 rotates to move the screw shaft 36 downward. Then, the plate 40 presses the packing 7a to make the communication tube 8b penetrate a thin film 7b and make the communication tube 8b of the ejection head portion 8 connected to the reservoir 7. After the connection, a rotary encoder 39 controls a moving distance of the screw shaft 36 and makes the plate 40 charge a specified amount of a medicine into an ejection head 8a. FIG. 8 illustrates the state in which the medicine has been completely charged.

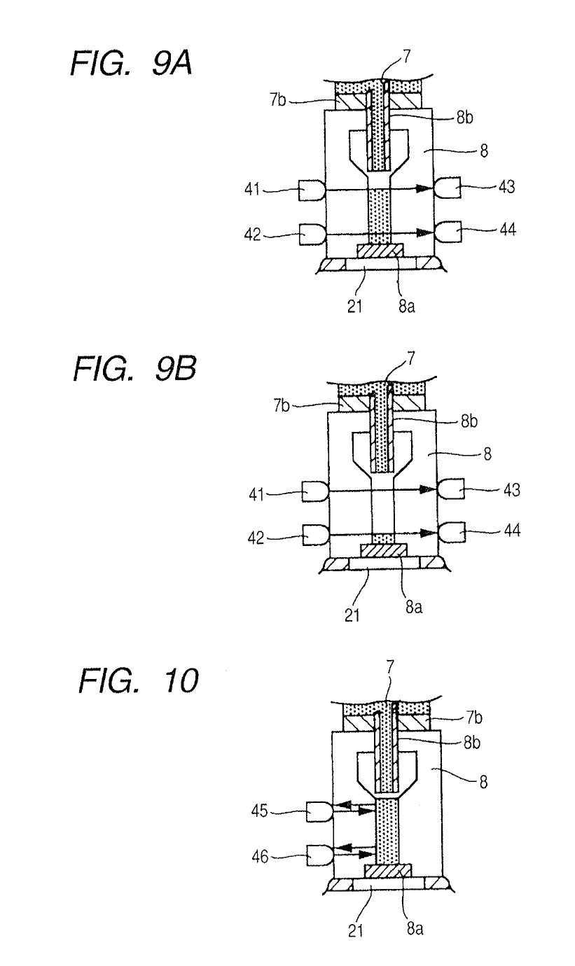

A level of a medicine gradually descends from the ejection starting state in FIG. 8. Then, a signal light emitted from a projector (first level) 41 arrives at an optical receiver (first level) 43. The state at the time is illustrated in FIG. 9A. As the inhaler continues ejection, the level of the medicine further descends. Then, a signal light emitted from a projector (second level) 42 arrives at an optical receiver (second level) 44. The state at the time is illustrated in FIG. 9B.

These projectors and optical receivers can be replaced with a reflective projector and optical receiver (first level) 45 and a projector and optical receiver (second level) 46 as are illustrated in FIG. 10, if they would sufficiently provide a signal-to-noise ratio (S/N ratio).

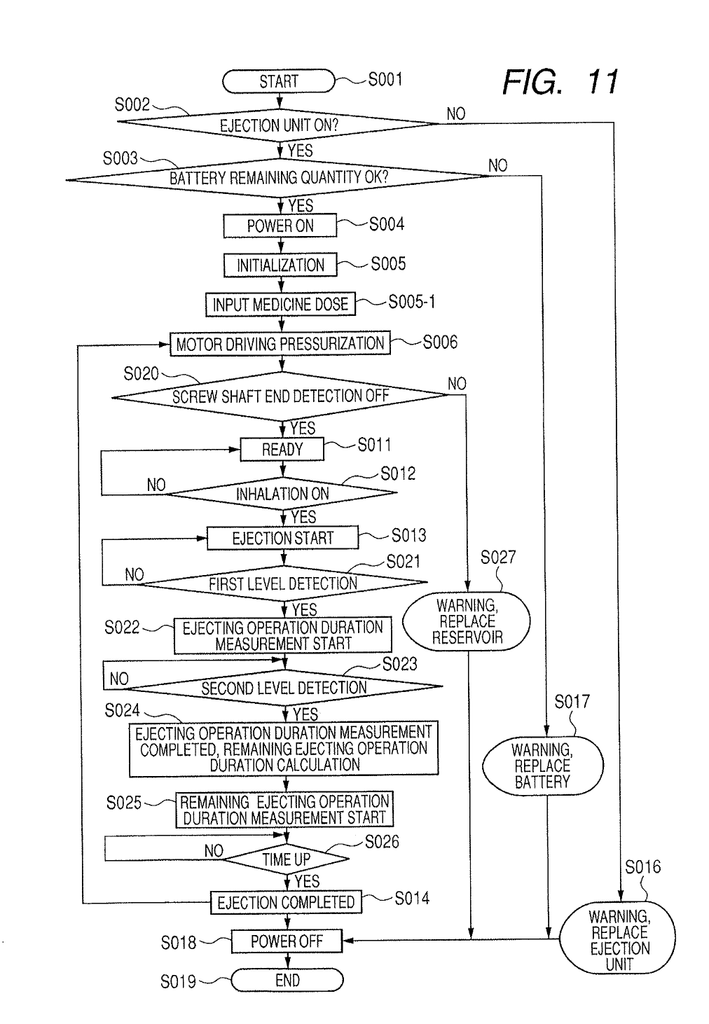

An action of the inhaler according to the present embodiment will be now described with reference to the flow chart illustrated in FIG. 11.

At first, the inhaler is set at a state of being capable of starting to be used by an operation of turning an electric power switch on by a user (S001: START). After the starting state, the inhaler examines whether a medicine ejection unit 6 is inserted therein or not (S002: EJECTION UNIT ON?). When the medicine ejection unit 6 is not inserted therein, the inhaler displays an alarm for informing the user the absence of the medicine ejection unit 6 (S016: WARNING, REPLACE EJECTION UNIT), turns the power source off (S018: POWER OFF), and finishes its action (S019: END).

When a medicine ejection unit 6 employs, for instance, a thermal jet type for ejecting a medicine, a method of measuring an ohmic value of a heater which is an ejection energy generation unit can be employed as a detecting unit of the medicine ejection unit 6.

When the detecting unit has detected the presence of a medicine ejection unit 6, the inhaler checks a remaining quantity of a battery in the main body of the device (S003: BATTERY REMAINING QUANTITY OK?). When the remaining quantity is short, the inhaler displays a message for urging the replacement or charging of the battery (S017: WARNING, REPLACE BATTERY), turns the power source off (S018: POWER OFF), and finishes its action (S019: END). When the inhaler has determined that the remaining quantity of the battery is sufficient at least for one inhaling action, the user turns the power source on (S004: POWER ON), and sets initial conditions (S005: INITIALIZATION).

After having finished setting the initial conditions, the user may be required to input a dose by oneself (S005-1: INPUT MEDICINE DOSE). Normally, the dose in a prescription data by a doctor is automatically set in the inhaler, but the user may change the does, for instance, of insulin in consideration of a caloric intake and calorie consumption when the user inhales the medicine.

An elevation compression motor 33 drives a motor gear 34, a driven gear 35 and a screw shaft 36 in such a direction as to move a reservoir 7 to an ejection head 8a side. Then, the reservoir 7 moves to the ejection head 8a side. When the reservoir 7 moves, a connection tube 8b penetrates a thin film 7b stuck on the reservoir 7, and the reservoir 7 starts charging a medicine into the ejection head portion 8. The elevation compression motor 33 further moves to charge the medicine into a nozzle of the ejection head 8a, which is a charging step. A rotary encoder 39 monitors the number of revolution of the motor, and determines that a liquid medicine with a predetermined amount of a dose has been supplied to a liquid ejection unit 6, namely, that the ejection head 8a has been completely filled with the medicine (S006: MOTOR DRIVING PRESSURIZATION). While the motor is rotating, the inhaler checks the presence or absence of a rotation completion detecting signal of the screw shaft 36 (S020). When the detecting signal turns on, the motor cannot rotate the screw shaft any more. Accordingly, the inhaler displays warning that the reservoir 7 is empty to inform the fact to the user (S027: WARNING, REPLACE RESERVOIR), turns the power source off (S018; POWER OFF), and finishes its action (S019: END). When the rotation completion detecting signal of the screw shaft 36 is off, the inhaler is ready for an inhalation, namely, is in a ready state (S011: READY). When a user turns the inhalation switch on (S012: INHALATION ON), the inhaler starts ejecting the medicine from the ejection head 8a (S013: EJECTION START).

In the next place, a step of measuring a medicine ejecting speed will be described.

The inhaler starts ejecting the medicine (S013: EJECTION START). As the medicine gradually decreases, the first level is detected at first (S021: FIRST LEVEL DETECTION). The inhaler measures the detected time (S022: EJECTING OPERATION DURATION MEASUREMENT START). As the medicine further decreases, the second level is detected (S023: SECOND LEVEL DETECTION). The inhaler measures the detected time (S024: EJECTING OPERATION DULATION MEASUREMENT COMPLETED, REMAINING EJECTING OPERATION DURATION CALCULATION). A medicine ejecting amount per unit time between the detected times can be calculated by measuring a period of time necessary for the level to move from the first level to the second level, because a medicine volume contained in between the first level and the second level of a medicine ejection unit 6 has been known, which are illustrated in FIGS. 9A and 9B. The medicine volume contained in between the second level and the surface of the ejection head 8a has been known beforehand, so that the rest of ejecting operation duration is calculated by dividing the medicine volume contained in between the second level and the surface of the ejection head 8a by the above described calculated medicine ejecting amount per unit time. Thereby, the inhaler can control an ejection unit so that the unit can compensate an error on the medicine ejecting amount, specifically, can keep the total ejection amount constant.

Subsequently, the inhaler starts measuring the rest of ejecting operation duration (S025: REMAINING EJECTING OPERATION DURATION MEASUREMENT START), and determines whether time is up or not (S026: TIME UP). When having determined that time is up, the inhaler finish the ejection (S014: EJECTION COMPLETED); stores a medicine remaining amount and a medicine ejection record (ejection time of day and ejected amount) in a flash ROM on a control substrate 18, turns a power source off (S018: POWER OFF), and finishes its action (S019: END).

A clock function in a CPU of a control substrate 18 undertakes a job of a time measurement portion (timer) which measures a period of transition time between the first level and the second level, and controls a driving period of time of an ejection head (ejecting operation duration). The above action is similarly carried out in Embodiment 1 as well.

There are variations of a pulmonary capacity among individuals of children, old men, sex and races. Accordingly, it is important to adjust a distance between the first level and the second level so that the inhaler completes the measurement in one inhalation of a user. When an inhalation detecting signal does not exceed a preset sensing level, in other words, the user cannot sufficiently inhale the medicine, the inhaler stops the inhalation. When the inhalation is not completed in one time, it can be separated into several times. In this case, the inhaler ejects an amount of the medicine corresponding to the rest of ejecting operation duration.

In the next place, the embodiment will be described with the use of specific numeric values. The total dose was set at 21 μL. An initial drive condition was set so as to eject the medicine with a drive frequency of 10 kHz. A volume of the medicine contained between the first level and the second level was 10 μL. The inhaler ejected the medicine, and took 1.0 sec for the surface of the medicine to move from the first level to the second level. Accordingly, the medicine ejecting amount per unit time while the surface of the medicine moved from the first level to the second level was calculated to be 10 μL/sec. The volume of the medicine contained in between the second level and the surface of the ejection head 8a was 6 μL, so that the rest of ejecting operation duration was calculated to be 6/10=0.6 sec. The controller drove the ejection head for 0.6 seconds after the medicine passed thorough the second level. As the result, the inhaler could surely eject the total dose with high accuracy.

The inhaler according to the present embodiment can surely eject a proper amount of the medicine even when one part of a nozzle is clogged after the ejection head 8a is used several times and a medicine ejecting amount per unit time deviates along with time, by adjusting an ejecting operation duration. The above medicine ejecting amount per unit time is occasionally referred to as “ejection speed”.

In order to secure a predetermined ejection amount, not only an ejecting operation duration can be adjusted, but also an ejection frequency, an ejection pulse width, a parameter (drive condition) of a drive voltage can be changed. In the above description, “ejection frequency” corresponds to the number of pulse signals per unit time, which are applied to an ejection pressure generating element so as to eject a medicine. In addition, “pulse width” is a current-carrying period of time in applied one pulse signal. As the pulse width increases, the amount of the medicine ejected during one pulse signal increases. It is also acceptable to determine a drive condition for achieving a necessary dose by changing a plurality of these combined methods. However, it is a simple method to adjust ejecting operation duration, because it may be limited by a capacity of each device to adjust an ejection frequency and the like.

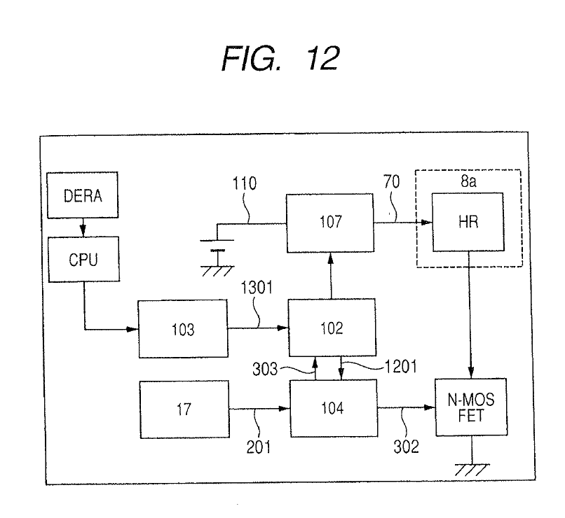

FIG. 12 illustrates a view of a circuit configuration of an ejection correction portion which has a drive condition readout circuit 102 and a drive condition table 103. The drive condition table 103 is a table for correcting a drive voltage, a pulse width and a frequency with respect to a remaining amount to be ejected. As any value of the drive voltage, the pulse width and the frequency increases, a correction ejection amount increases.

When a driving pulse control circuit 104 receives a command to start ejection correction, the control circuit 104 sends a drive condition requesting signal 303 to a drive condition readout circuit 102 and requests to send a drive condition. The drive condition requesting signal 303 is a signal of a logic level, which is true after a new drive condition becomes necessary and before the drive condition is set.

The drive condition readout circuit 102 sequentially reads out drive condition data 1301 from a drive condition table 103 every time of receiving a drive condition requesting signal 303, and sets a drive condition in the driving pulse control circuit 104 through a drive condition setting signal 1201.

When a drive condition is set on a driving pulse control circuit 104 by the drive condition setting signal 1201, the control circuit 104 temporarily withdraws the drive condition requesting signal 303, and controls a drive pulse signal 302 on the basis of a set drive condition to make an ejection head 8a eject a medicine.

A drive condition to be set in a driving pulse control circuit 104 includes the ON period condition and OFF period condition of a drive pulse signal 302, ON/OFF repeated times and a continuation/ending flag. The driving pulse control circuit 104 repeats ON and OFF of the drive pulse signal 302 by a commanded number of repeating times in the set ON period and OFF period. When the continuation/ending flag shows continuation after the driving pulse control circuit 104 has finished controlling the drive pulse signal 302 by the commanded number of repeating times, the control circuit 104 requests the drive condition readout circuit to send a new drive condition through the drive condition requesting signal 303. When the continuation/ending flag shows ending, the driving pulse control circuit 104 does not request a new condition but finishes ejection control. In FIG. 12, the reference numeral 107 denotes a driving voltage control circuit, and the expressions “DERA” and “HR” means “detecting ejection remaining amount” and “heating resistor”, respectively.

An ejection head mounted on the inhaler uses thermal energy in any of the above described embodiments, but it goes without saying that the ejection head can use piezoelectric energy. The inhaler may also have such a configuration as to make a measurement portion measure a remaining amount of a medicine in a reservoir and inform the remaining amount of the medicine to the user. Then, the user can use the inhaler with peace of mind; can reduce a preliminary ejection amount wasted for restoring the ejection properties; can efficiently inhale the medicine in the reservoir; and consequently can also reduce an economic burden of the user.

A medicine ejection device according to the present invention can be applied to not only an inhaler for a medicine but also a device for ejecting a flavoring agent or the like in a form of mist and an inhaler of luxury goods such as nicotine.

The present invention is not limited to the above embodiments and various changes and modifications can be made within the spirit and scope of the present invention. Therefore to apprise the public of the scope of the present invention, the following claims are made.

This application claims the benefit of Japanese Patent Application No. 2006-209365, filed Aug. 1, 2006 and No. 2007-193998, filed Jul. 26, 2007, which are hereby incorporated by reference herein in their entirety.

1. A medicine ejection device for ejecting a medicine to be inhaled by a user comprising:
a medicine ejection portion for ejecting the medicine;
a measurement portion for measuring an amount of the medicine ejected from the medicine ejection portion; and
a controller for driving the medicine ejection portion so as to eject the amount of the medicine, which corresponds to a difference between the amount of the medicine to be administered and the amount of the ejected medicine, on the basis of a value measured in the measurement portion.

2. The medicine ejection device according to claim 1, characterized in that the measurement portion measures the medicine ejecting amount by measuring a remaining amount of the medicine in a reservoir for storing the medicine with the use of remaining amount measuring means.

3. The medicine ejection device according to claim 1, characterized in that the measurement portion comprises optical means for measuring an amount of the medicine per unit time ejected from the medicine ejection portion.

4. The medicine ejection device according to claim 1, characterized in that the controller adjusts a drive condition including an ejection frequency of the medicine ejection portion, an ejection pulse width, a drive voltage or an ejecting operation duration.

5. The medicine ejection device according to claim 1, characterized in that the controller adjusts the ejecting operation duration of the medicine ejection portion in the following inhalation on the basis of the measured value in the measurement portion when the user has inhaled the medicine one time.

6. The medicine ejection device according to claim 1, characterized in that the controller adjusts the ejecting operation duration of the medicine ejection portion in one inhalation on the basis of the measured value in the measurement portion when the user has inhaled the medicine one time.

7. The medicine ejection device according to claim 1, characterized in that the medicine ejection portion has an electrothermal transducer for applying heat energy to the medicine or an electromechanical transducer for applying mechanical energy to the medicine.
